(19) United States
(12) Patent Application Publication
(10) Pub. No.: US 2020/0255305 A1
(43) Pub. Date: Aug. 13, 2020

(54) METHOD AND DEVICE FOR WATER DISINFECTION

(71) Applicant: Ramot at Tel-Aviv University Ltd., Tel-Aviv (IL)

(72) Inventors: Yoram Gerchman, KiryatTivon (IL); Hadas Mamane, Caesarea (IL); Vered Cohen Yaniv, Tel-Aviv (IL); Yifaat Betzalel, Tel-Aviv (IL); Avraham Balas, Tel-Aviv (IL)

(21) Appl. No.: 16/481,422

(22) PCT Filed: Feb. 15, 2018
(86) PCT No.: PCT/IL2018/050174

§ 371 Date: Jul. 26, 2019

Related U.S. Application Data
(60) Provisional application No. 62/459,228, filed on Feb. 15, 2017.

(51) Int. Cl. C02F 1/32

(52) U.S. Cl. CPC C02F 1/325; C02F 2201/3227; C02F 2201/3222; C02F 2303/04

(57) ABSTRACT

The present invention provides a method for water disinfection by exposing water, optionally simultaneously, to a combination of at least two ultraviolet (UV) irradiation sources, wherein at least one of said UV irradiation sources emits light at a wavelength of between 250 nm and 280 nm, and at least one of said UV irradiation sources emits light at a wavelength of between 285 nm and 310 nm; and an apparatus for carrying out said method.

### TECHNICAL FIELD

The present invention provides a method for water disinfection, and a water disinfection apparatus for carrying out said method.

### BACKGROUND ART

Ultraviolet (UV) irradiation is a well-established practice for water disinfection of pathogens. The UV spectrum is divided into UVC (200-280 nm), UVB (280-315 nm), and UVA (315-400 nm). Currently, two types of mercury vapor-filled lamps produce germicidal UV irradiation which are either nearly monochromatic at about 254 nm (253.7 nm) (low pressure/low pressure high output, LP/LPHO), or polychromatic (medium pressure, MP) that emit light at multiple peaks from 200 nm and above, including at 254 nm. Full scale water disinfection UV-based systems vary in size, configuration (horizontal or vertical to the flow), lamp type (LP or MP), UV transmittance (UVT) sensors, and reactor design (in-line reactors or submerged lamps in open conduit) (Water Environment Federation, 2015).

Germicidal UV action refers to the wavelengths of UV irradiation that induce photochemical reactions effective in microbial inactivation. Nucleic acids show significant absorption of UV photons between 200 and 300 nm, with peak absorption at 265 nm. A UV action spectrum is determined by measuring the dose response of a microorganism to various wavelengths. This action spectrum highly depends on the wavelength and may vary between different microorganisms. The absorbance spectrum of an isolated DNA of a particular microorganism may differ from the actual action spectrum, because photons can be absorbed in other cell components as shown for Bacillus subtilis spores (Chen et al., 2009; Mamane-Gravetz et al., 2005). Thus, calculating a MP UV average germicidal dose based on DNA absorption (typically determined for Escherichia coli; Meulemans, 1987), with relative peak sensitivities in the 260-265 nm region, does not account for non-DNA-based damages.

The power input of LP lamps or LPHO measured per lamp (100 W and 150-500 W, respectively) is lower than for MP lamps (3000-5000 W), and the number of LP lamps required so as to provide a similar wattage is thus higher. Nevertheless, the germicidal efficiency, i.e., the conversion efficiency of electrical power to UVC photons in the absorbance spectrum of DNA for LP lamps, is higher than that for MP lamps, and therefore more electrical energy is required by MP lamps to emit the same UV energy as LP lamps.

MP lamps are disadvantageous over LP/LPHO lamps because they are more expensive; have lower germicidal efficiency (12-16%) compared to LPHO (30-35%); have higher operating temperature (600-950° C.) compared to LPHO (150-200° C.), which results in higher fouling rate on the lamp sleeves (removal of the fouling requires cleaning by mechanical (wipers) and chemical (acid) methods and disposal of chemical waste, thus increasing the maintenance cost); have a shorter life time (8000 hours) compared to LP/LPHO (12,000-16,000 hours); and emit wavelengths that are non-usable for disinfection, e.g., in the infrared (IR) and longer warm up time. However, MP lamps have still advantageous considering that they target cellular components other than the DNA, such as proteins (various proteins and enzymes were found to absorb UVB and UVC, resulting in further bacterial damage (Harm, 1980; Oguma et al., 2002; Sinha and Hader, 2002). Proteins typically show an absorption peak around 280 nm, with a minimum around 240-250 nm (Jagger, 1967); and that less lamps are required compared to LP so as to achieve the same effect.

As stated above, the germicidal action spectrum of a UV light may depend on the microorganism. For example, while organisms such as E. coli, Salmonella typhimurium, Pseudomonas aeruginosa, B. subtilis, Cryptosporidium parvum oocysts, and Bacillus pumilus have relative peak sensitivities in the range of 260-265 nm (Beck et al., 2015; Chen et al., 2009; Gates, 1930; Lakretz et al., 2010; Linden et al., 2001; Mamane-Gravetz et al., 2005; Wang et al., 2005), Herpes simplex virus exhibits peak sensitivities in the range of 270-280 nm (Detsch et al., 1980). The maximum sensitivity of various microorganisms thus extends beyond the 254 nm emitted by LP lamps. An additional issue is the bacterial recovery following exposure to mercury UV lamps Zimmer and Slawson (2002) showed that E. coli underwent photorepair following exposure to LP, but no repair was detected following exposure to the MP lamp (10 mJ/cm²).

MP systems are typically equipped with automatic wipers to address fouling of quartz sleeves. Occasionally, the automatic cleaning is not sufficient, and this results in increase in the intensity of the lamps (the UV sensor senses a decrease in lamp intensity and thus increases the intensity of the lamp to compensate), and consequently in increase in the system electrical consumption and decrease in the disinfection effectiveness. The above results in temperature increase followed by inorganic precipitation, up to a situation where the transmitted irradiation is so low that the system must be disabled to perform chemical acid cleaning. Precipitation of the MP lamp sleeve is more severe compared to LPHO lamp, as MP lamp temperature reaches 950° C. compared to 150-200° C. in LPHO, and chemical acid cleaning for MP lamps is therefore required more frequently than for LPHO lamps. Accordingly, the US Environmental Protection Agency (USEPA) (2006) guidelines for UV specifically recites: “Compounds for which the solubility decreases as temperature increases may precipitate (e.g., CaCO₃, CaSO₄, MgCO₃, MgSO₄, FePO₄, FeCO₃, Al₂(SO₄)₃) and foul MP lamps faster than LP or LPHO lamps because MP lamps operate at higher temperatures”.

UV light-emitting diodes (LEDs) are considered as alternatives to UV mercury lamps in water treatment. These UV sources allow flexible design (point source vs. cylindrical tube in LP and MP) and construction of UV reactors, and enables tuning the wavelength; require no lamp warm-up time; can be operated by intermittent flow and at ambient temperature, thus do not promote fouling; have lower electricity consumption; and require a DC voltage thus can be powered with a battery/solar cell. In contrast to mercury lamps, LEDs are safe to dispose (Chen et al., 2017).

UVC-LEDs have shown ability to inactivate bacteria as E. coli, spores of B. subtilis and bacteriophages (MS2) (Chen et al., 2017). However, this technology is currently limited due to low power input and radiant flux, which limit the efficiency to a few percentages only, short life-time, and extremely high costs, and it is therefore immature for industrial use in water disinfection.

### SUMMARY OF INVENTION

In one aspect, the present invention relates to a method for water disinfection comprising exposing water, optionally simultaneously, to a combination of at least two UV irradiation sources, for a sufficient period of time, wherein at least one of said UV irradiation sources emits light at a wavelength of between 250 nm and 280 nm, and at least one of said UV irradiation sources emits light at a wavelength of between 285 nm and 310 nm. In particular embodiments, said at least one UV irradiation source emitting light at a wavelength of between 250 nm and 280 nm comprises a LP UV irradiation source emitting light at a wavelength of about 254 nm, or a UV LED irradiation source emitting light at a wavelength of between 260 nm and 270 nm; and said at least one UV irradiation source emitting light at a wavelength of between 285 nm and 310 nm comprises a UV LED irradiation source emitting light at a wavelength of between 290 nm and 300 nm.

In another aspect, the present invention provides a water disinfecting apparatus comprising: (i) a chamber having an inlet adapted for connection to a pressurized water source, and an outlet; (ii) at least one UV irradiation source emitting light at a wavelength of between 250 nm and 280 nm; and (iii) at least one UV irradiation source emitting light at a wavelength of between 285 nm and 310 nm, wherein said UV irradiation sources are designed/configured to emit UV light into said chamber when water passes therethrough. Particular such apparatus are those wherein said at least one UV irradiation source emitting light at a wavelength of between 250 nm and 280 nm comprises a LP UV irradiation source emitting light at a wavelength of about 254 nm, or a UV LED irradiation source emitting light at a wavelength of between 260 nm and 270 nm; and said at least one UV irradiation source emitting light at a wavelength of between 285 nm and 310 nm comprises a UV LED irradiation source emitting light at a wavelength of between 290 nm and 300 nm.

### BRIEF DESCRIPTION OF DRAWINGS

FIG. 1 shows spectral emission of MP and LP UV lamps.

FIGS. 2A-2B show a schematic diagram of the LP-LED collimated beam apparatus described in the Experimental setup below, including a mercury LP lamp and a LED array, and an electromagnetic peripheral source for stirring the water sample (2A); and the stirring device designed to allow effective mixing of the water sample irradiated (2B).

FIG. 3 shows SodA gene activation in E. coli by exposure to MP lamp (right); and the plasmid with lux genes fused to the SodA promoter used (left).

FIG. 4 shows E. coli germicidal inactivation by LP and MP lamps, where the average irradiation was compared and weighted between wavelengths of 220-300 nm and 250-262 nm (the range in which the two lamps emit).

FIG. 5 shows log inactivation of E. coli MG1655 strain vs. irradiation time, by a UV LED irradiation source emitting light in the 285-305 nm range, a LP irradiation source emitting light of about 254 nm, and the combination thereof, compared to the theoretical value representing the LP+LED summation.

FIG. 6 shows log inactivation of E. coli MG1655 strain vs. germicidal dose, by a combination of LP+UV LED irradiation source emitting light in the 285-305 nm range, compared to the theoretical value representing the LP+LED summation.

FIG. 7 shows log inactivation of natural indigenous coliforms in wastewater secondary effluent by LP or LP+UV LED in the 285-305 nm range.

FIG. 8 illustrates a water disinfecting apparatus according to one embodiment of the invention, having a tube/cylindered-shaped chamber.

FIG. 9 illustrates a cross-section view of the water disinfecting apparatus illustrated in FIG. 8, showing the location of the inner LP UV lamp along the centerline radius of the tube, while the LED UV lamps are positioned around and across the perimeter of said tube.

FIG. 10 illustrates a cross-section view of the water disinfecting apparatus illustrated in FIG. 8, and further illustrates that the surroundings of the transparent regions at the tube are designed to enable easy mounting and replacing of an individual LED array.

FIG. 11 illustrates various ultraviolet reactor baffle designs for advanced ultraviolet disinfection showing various ways to increase turbulence of the water passing within the tube.

### DETAILED DESCRIPTION

It has now been found in accordance with the present invention that while photons from LP UV lamp target mainly DNA, resulting in formation of nucleotide dimers, irradiation of bacteria with photons at wavelengths in the UVB spectrum, more specifically between 285 nm and 305 nm, results in the production of superoxide radicals inside the bacterial cell, most likely due to photoactivation of aromatic amino acids and mainly tryptophan. Such radicals can target cellular components other than DNA, e.g., cell membrane, small molecules, and proteins that are essential for almost all cell activities, including the repair of DNA dimers.

As shown herein, the combination of DNA damaging photons, i.e., photons emitted from an irradiation source emitting light at a wavelength in the range of 250-280 nm, e.g., a LP UV lamp emitting light at a wavelength of about 254 nm or a UV LED emitting light at a wavelength in the range of 260-270 nm; with superoxide generating photons, i.e., photons emitted from an irradiation source emitting light at a wavelength of 285-310 nm, e.g., UVB LED lamp, provides a synergistic effect and results in much more effective inactivation of pathogens and lower recovery thereof. For example, a combination of a LP UV lamp emitting light at a wavelength of about 254 nm and a UV LED emitting light at a wavelength of 285-305 nm resulted in enhanced and synergistic inactivation of E. coli strains compared to either one of said irradiation sources and compared to the theoretical value representing the LP+LED summation, wherein germicidal efficiency is increased exponentially with increase in the irradiation time.

While MP UV lamps require much more electricity and produce much more heat, resulting in precipitation of scaling on the quartz sleeve covering the lamp and the need for periodic complex and expensive cleaning, a combined system as shown herein will have both the benefits of LP/LPOH, i.e., low power consumption and low heat production, and of MP systems, i.e., better inactivation, and less recovery; and could be used for inactivation of microorganisms in a variety of applications, including water and air disinfection.

A combined apparatus as disclosed herein will thus achieve MP level inactivation of waterborne pathogens while avoiding the disadvantages of current MP lamps (Table 1), wherein careful selection of the LEDs wavelength(s) could be chosen to damage the pathogen by additional mechanisms (production of superoxide radicals damaging cellular components other than the DNA, etc.) resulting in a synergistic effect. Furthermore, the modular design of the apparatus could offer easy tailoring of the apparatus (and the method using said apparatus) for specific pathogens by choosing the required LEDs and replacing them as needed.

In one aspect, the present invention thus relates to a method for water disinfection comprising exposing water, optionally simultaneously, to a combination of at least two UV irradiation sources, for a sufficient period of time, wherein at least one of said UV irradiation sources emits light at a wavelength of between 250 nm and 280 nm, and at least one of said UV irradiation sources emits light at a wavelength of between 285 nm and 310 nm.

The phrase “UV irradiation source emitting light at a wavelength of between 250 nm and 280 nm” means a monochromatic (or nearly monochromatic) UVC irradiation source having a definite wavelength between 250 nm and 280 nm, or a polychromatic UVC irradiation source with emission wavelength, but preferably with peak emission wavelength, between 250 nm and 280 nm.

The phrase “UV irradiation source emitting light at a wavelength of between 285 nm and 310 nm” means a monochromatic (or nearly monochromatic) UVB irradiation source having a definite wavelength between 285 nm and 310 nm, or a polychromatic UVB irradiation source with emission wavelength, but preferably with peak emission wavelength, between 285 nm and 310 nm.

TABLE 1
Comparison between LP/LPOH, MP, UV-LED and the system disclosed herein

| Lamp type | LP | LPHO | MP | | UV-LED | Suggested hybrid system |
|---|---|---|---|---|---|---|
| Wavelength | Monochromatic | | Polychromatic | | Wavelength tuned | Multichromatic and |
| | 254 nm | | germicidal 200-300 nm | | | wavelength tuned |
| Operating temp | 30-50° C. | 150-200° C. | 600-950° | C. | Same as process water | Same as LP |
| Germicidal | 35-40% | 30-35% | 12-16% | | Up to a few | 45-50% (for UVA |
| efficiency | | | | | percentages for UVC | LED) |
| Lamp life | 12,000-16,000 hrs | | 8,000 | hrs | 1,000 hrs for UVC | >20,000 hrs (for UVA |
| | | | | | | LED) |
| Power input | ~100 W | 150-500 W | 3000-5000 | W | Low, up to a few Watts | Up to 600 W |
| Fouling | Low | | High | | No fouling | Same as LP |
| Warm up time | 2 min | 5 min | 10 | min | Instantaneous | Same as LP |

| Mercury | 20-200 mg | | Safe disposal | Same as LP |
|---|---|---|---|---|
| content | | | | |
| Architecture | Cylindrical tube | | Point source | Same as LP |
| Shell material | Quartz | | | Quartz for LP, |
| | | | | polymer for LED |
| Advantages | Low cost, high germicidal | Less lamps needed compared to LP | Intermittent flow | Same as LP plus |
| | efficiency, low fouling, | (higher power output), target | friendly, point of use, | benefits of MP-multi |
| | less maintenance, low | cellular components other than | small, versatile, no fouling, | wavelength and better |
| | temperature, lifetime | DNA and thus more effective in | targeted performance, long | disinfection |
| | longer than MP, point of | certain cases, recovery is | life time | |
| | use systems | controlled | | |
| Disadvantages | Low power output and more | Expensive, germicidal efficiency | Very high cost for UVC | |
| | lamps needed, recovery | lower than that of LP/LPHO, high | LED, lower cost for UVA | |
| | issues, may be less | operating temperature resulting | LED, low power input (many | |
| | effective in disinfection | more fouling and required sleeve | LEDs), UVC LEDs not used | |
| | (depending on integration | cleaning with mechanical | commercially | |
| | method and pathogen type) | (wipers) and chemical (acid), | | |
| | | require disposing chemical waste | | |
| | | due to acid cleaning, short life | | |
| | | time, long warm-up time | | |

In certain embodiments, said at least one UV irradiation source emitting light at a wavelength of between 250 nm and 280 nm and utilized according to the method of the present invention comprises a sole UV irradiation source, or a plurality of UV irradiation sources emitting light at either the same or different wavelengths between 250 nm and 280 nm. In certain particular such embodiments, said at least one UV irradiation source comprises a low pressure (LP) UV irradiation source, e.g., a LP UV irradiation source emitting light at a wavelength of about 254 nm. In other particular such embodiments, said at least one UV irradiation source comprises at least one UV light-emitting diode (LED) irradiation source each independently emitting light at a wavelength of between 260 nm and 275 nm, or between 260 nm and 270 nm, e.g., at a wavelength of about 260 nm, about 261 nm, about 262 nm, about 263 nm, about 264 nm, about 265 nm, about 266 nm, about 267 nm, about 268 nm, about 269 nm, or about 270 nm.

In certain embodiments, said at least one UV irradiation source emitting light at a wavelength of between 285 nm and 310 nm and utilized according to the method of the present invention comprises a sole UV LED irradiation source or a plurality of UV LED irradiation sources/array emitting light at either the same or different wavelengths between 285 nm and 310 nm. In particular such embodiments, each one of said UV LED irradiation sources independently emits light at a wavelength of between 285 nm and 290 nm, between 290 nm and 295 nm, between 295 nm and 300 nm, between 300 nm and 305 nm, or between 305 nm and 310 nm, e.g., at a wavelength of about 290 nm, about 291 nm, about 292 nm, about 293 nm, about 294 nm, about 295 nm, about 296 nm, about 297 nm, about 298 nm, about 299 nm, or about 300 nm.

According to the method of the present invention, the water treated, i.e., undergoing disinfection, are exposed, for a sufficient period of time, to a combination of at least two UV irradiation sources as defined in any one of the embodiments above. In certain embodiments, the exposure of said water to said at least two UV irradiation sources is carried out sequentially at any order; and in other embodiments, the exposure of said water to said at least two UV irradiation sources is carried out simultaneously.

The phrase “sufficient period of time” as used herein means a period of time that is sufficient to disinfect the water treated according to the method of the invention. Such a period of time may vary depending on various parameters such as the type/nature of the water treated (drinking water, wastewater effluents of different origins, etc.); water quality parameters including UV transmittance (UVT); water flow rate; the microbial pathogens characterizing, i.e., found in, said water; geographic-related conditions; and designated use. A period of time sufficient for disinfecting the water treated by this method may range from a few seconds, e.g., 1, 2, 3, 4, 5, 6, 7, 8, 9 or 10 seconds, and up to a few minutes, e.g., 1, 2, 3, 4, 5 or 6 minutes, but it preferably ranges from a few seconds and up to several tens of seconds, i.e., up to 10, 20, 30, 40, 50, 60, 70, 80, 90, 100, 110 or 120 seconds. Yet, it may be assumed that the period of time required for disinfecting said water, when exposed to said at least two UV irradiation sources sequentially, would be longer than that required when said water are exposed to said UV irradiation sources simultaneously.

In certain embodiments, the present invention relates to a method for water disinfection as defined in any one of the embodiments above, wherein (i) said at least one UV irradiation source emitting light at a wavelength of between 250 nm and 280 nm comprises a LP UV irradiation source emitting light at a wavelength of about 254 nm; or a UV LED irradiation source emitting light at a wavelength of between 260 nm and 275 nm, or between 260 nm and 270 nm, e.g., at a wavelength of about 260 nm, about 261 nm, about 262 nm, about 263 nm, about 264 nm, about 265 nm, about 266 nm, about 267 nm, about 268 nm, about 269 nm, or about 270 nm; and (ii) said at least one UV irradiation source emitting light at a wavelength of between 285 nm and 310 nm comprises a UV LED irradiation source emitting light at a wavelength of between 285 nm and 290 nm, between 290 nm and 295 nm, between 295 nm and 300 nm, between 300 nm and 305 nm, or between 305 nm and 310 nm, e.g., at a wavelength of about 290 nm, about 291 nm, about 292 nm, about 293 nm, about 294 nm, about 295 nm, about 296 nm, about 297 nm, about 298 nm, about 299 nm, or about 300 nm. In particular such embodiments, the exposure of said water to said at least two UV irradiation sources is carried out simultaneously.

In another aspect, the present invention provides an apparatus 200, or system, for carrying out the method of the invention, more particularly, a water disinfecting apparatus 200 that enables simultaneous irradiation from two directions while mixing the passing water. This apparatus 200 comprises (i) a chamber 201, e.g., a vessel or reactor, having an inlet 202 adapted for connection 201 to a pressurized water source, and an outlet 203; (ii) at least one UV irradiation source 207 emitting light at a wavelength of between 250 nm and 280 nm; and (iii) at least one UV irradiation source emitting light at a wavelength of between 285 nm and 310 nm, wherein said UV irradiation sources are designed/configured to emit UV light into said chamber 201 when water passes therethrough.

In certain embodiments, the present invention provides a system as defined above, wherein said at least one UV irradiation source 207 emitting light at a wavelength of between 250 nm and 280 nm comprises a sole UV irradiation source 207 or a plurality of UV irradiation sources emitting light at either the same or different wavelengths between 250 nm and 280 nm. Particular such systems are those wherein said at least one UV irradiation source comprises a LP UV irradiation source 207, e.g., a LP UV irradiation source emitting light at a wavelength of about 254 nm. In other embodiments, the invention provides a system as defined above, wherein said at least one UV irradiation source comprises at least one UV LED irradiation source each independently emitting light at a wavelength of between 260 nm and 275 nm, between 260 nm and 270 nm, or between 260 nm and 265 nm.

In certain embodiments, the present invention provides a system as defined above, wherein said at least one UV irradiation source emitting light at a wavelength of between 285 nm and 310 nm comprises a sole UV LED irradiation source or a plurality/array of UV LED irradiation sources 205 emitting light at either the same or different wavelengths between 285 nm and 310 nm. In particular such embodiments, each one of said UV LED irradiation sources independently emits light at a wavelength of between 285 nm and 290 nm, between 290 nm and 295 nm, between 295 nm and 300 nm, between 300 nm and 305 nm, or between 305 nm and 310 nm.

In certain embodiments, the present invention provides a system as defined above, wherein (i) said at least one UV irradiation source 207 emitting light at a wavelength of between 250 nm and 280 nm comprises LP UV irradiation source emitting light at a wavelength of about 254 nm; or a UV LED irradiation source emitting light at a wavelength of between 260 nm and 275 nm, or between 260 nm and 270 nm, e.g., at a wavelength of about 260 nm, about 261 nm, about 262 nm, about 263 nm, about 264 nm, about 265 nm, about 266 nm, about 267 nm, about 268 nm, about 269 nm, or about 270 nm; and (ii) said at least one UV irradiation source emitting light at a wavelength of between 285 nm and 310 nm comprises a UV LED irradiation source 205 emitting light at a wavelength of between 285 nm and 290 nm, between 290 nm and 295 nm, between 295 nm and 300 nm, between 300 nm and 305 nm, or between 305 nm and 310 nm, e.g., at a wavelength of about 290 nm, about 291 nm, about 292 nm, about 293 nm, about 294 nm, about 295 nm, about 296 nm, about 297 nm, about 298 nm, about 299 nm, or about 300 nm.

The water disinfecting apparatus 200 of the present invention comprises a chamber 201 having an inlet 202 adapted for connection to a pressurized water source, and an outlet 203; as well as at least two UV irradiation sources each as defined in any one of the embodiments above, configured to emit UV light into said chamber 201 when water passes therethrough. Said chamber 201 may be configured, e.g., as a tube.

In certain embodiments, the system of the present invention comprises a chamber 201 configured as a tube, wherein (i) said at least one UV irradiation source 207 emitting light at a wavelength of between 250 nm and 280 nm is located across the center of said tube; and (ii) said at least one UV irradiation source emitting light at a wavelength of between 285 nm and 310 nm is a single UV LED or UV LED array(s) 205 located at the perimeter of said tube, e.g., distributed around and across the perimeter of said tube.

In particular such embodiments, said tube is either made of, or comprises at least one region 204 made of, a material transparent to said UV LED irradiation of a wavelength of between 285 nm and 310 nm; and said at least one UV LED irradiation source emitting light at a wavelength of between 285 nm and 310 nm is located externally to said tube or said at least one region 204, respectively, such that it does not come in contact with the water when passes through said tube. In yet other particular embodiments, said material transparent to said UV LED irradiation of a wavelength of between 285 nm and 310 nm is not transparent to UV irradiation of a wavelength of between 250 nm and 280 nm.

In other particular such embodiments, said UV LED irradiation source emitting light at a wavelength of between 285 nm and 310 nm is located inside said tube and is waterproof. In yet other particular such embodiments, said tube is made of opaque material or from material not transparent to the UV wavelengths emitted by either UV irradiation sources.

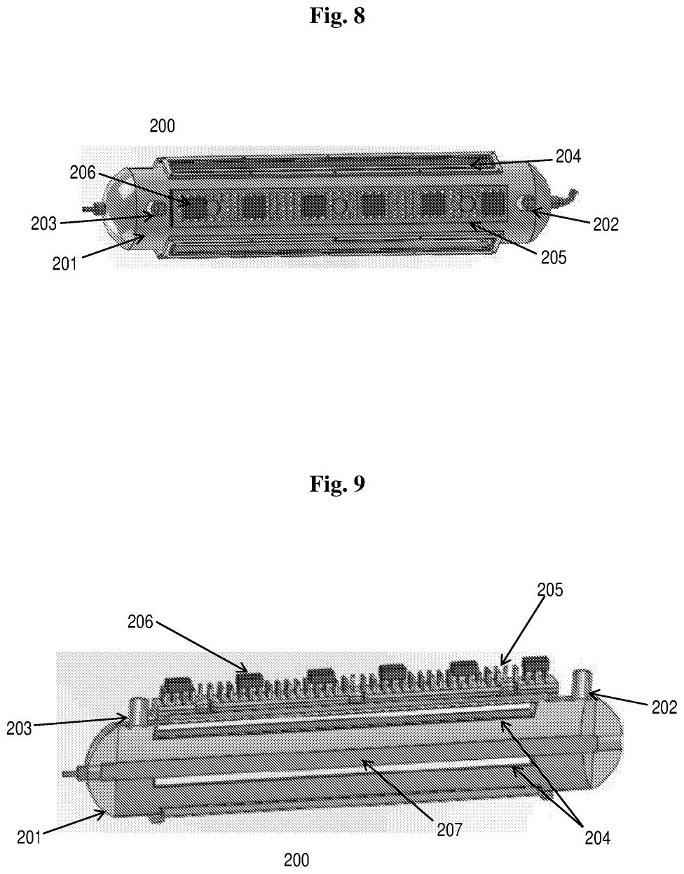

FIG. 8 shows an illustration of a water disinfecting apparatus 200 according to one embodiment of the invention, having a tube/cylindered-shaped chamber 201. FIG. 9 illustrates a cross-section view of the water disinfecting apparatus 200 illustrated in FIG. 8, showing the location of the inner LP UV lamp along the centerline radius of the tube, while the LED UV lamps/arrays 205 are positioned around and across the perimeter of said tube. Notably, FIGS. 8-9 illustrate a single LED array 205, but it should be noted that similar arrays are positioned/evenly-distributed all-around said tube. As illustrated in FIG. 8, the water disinfecting apparatus 200 of the invention may further comprise a cooling unit 206 for cooling the UV LED array 205 if needed or the water passing therein. Alternatively, the water disinfecting apparatus 200 of the invention may further comprise a heating unit, either instead or in addition to said cooling unit 206, for heating the water passing therein, e.g. for increasing the disinfection process.

Notably, when the entire tube is made of a transparent material, said LED arrays 205 can be positioned at any location at the perimeter of the tube, wherein the areas in between said LED arrays 205 might be covered in order to prevent UV light from exiting the tube. Such covering may be done with either a reflective material/surface (as detailed herein below), or a light-absorbent material. Alternatively, when the tube comprises transparent regions 204 (as illustrated in FIGS. 8-10), said LED arrays 205 are positioned over said transparent regions 204 to enable UV light emitted therefrom to reach the water when it passes through said tube.

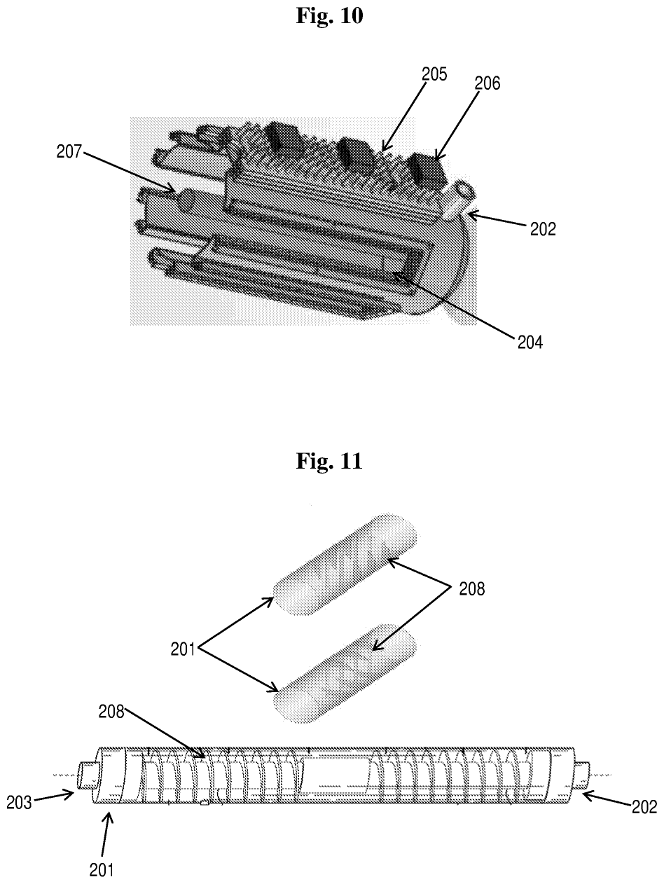

FIG. 10 illustrates another cross-section view of the water disinfecting apparatus 200 illustrated in FIGS. 8-9, further illustrating that the surroundings of the transparent regions 204 at the tube are designed to enable easy mounting and replacing of an individual LED array 205. As illustrated in FIG. 10, each LED array 205 and transparent region 204 may comprise rails and grooves for sliding said LED array 205 on said transparent region 204, and off when needed, e.g., for repair or changing the UV wavelength emitted by the LED array 205.

In further particular such embodiments, said at least one UV irradiation source 207 emitting light at a wavelength of between 250 nm and 280 nm is located in a UVC transparent sleeve located at the center along the axis of said tube, i.e., along the centerline radius. In more particular such embodiments, the transparent sleeve is made of quartz, soda lime glass (also called soda-lime-silica glass), or a UVC-transparent polymer such as a polyacrylate (commonly known as acrylics). Non-limiting examples of polyacrylates include polyacrylamide, polyacrylonitrile, polymethylacrylate, polymethylmethacrylate (known as plexiglass), polyethylacrylate, polypropylacrylate, poly(2-ethylhexyl)acrylate, polyhydroxyethylmethacrylate, polybutylacrylate, and sodium polyacrylaye.

In still further particular such embodiments, (i) said at least one UV LED irradiation source emitting light at a wavelength of between 285 nm and 310 nm is distributed around and across the perimeter of said tube, and is located externally such that it does not come in contact with the water when passes through said tube; and (ii) said at least one UV irradiation source 207 emitting light at a wavelength of between 250 nm and 280 nm is located in a transparent sleeve located at the center along the axis of said tube, wherein said transparent sleeve is made of quartz or a UVC-transparent polymer.

In certain embodiments, the water disinfecting apparatus 200 of the present invention comprises a chamber 201 configured as a tube, according to any one of the embodiments above, wherein said tube further comprises reflective surface(s) positioned onto the inner surface of said tube, for increasing the effect of UV irradiation on the water when passes through said tube, due to total reflection (and not absorbance) of the UV photons at the walls. In particular such embodiments, said reflective surface(s) are made of polytetrafluoroethylene (Teflon), aluminum, stainless steel, or a refractive polymer, optionally coated for better reflectance. In other particular such embodiments, said reflective surface(s) are made of Heraeus quartz materials such as HSQ100. In other embodiments, said reflective surfaces are positioned onto the outer surface of said tube, e.g., between the LED arrays 205.

In certain embodiments, the water disinfecting apparatus 200 of the present invention comprises a chamber 201 configured as a tube, according to any one of the embodiments above, wherein said tube further comprises baffles 208 to enhance radial mixing (whirling) of water when passes through said tube, and consequently increase the period during which said water is being exposed to said UV irradiation. Examples of a tube having such baffles 208 are illustrated in FIG. 11.

In certain embodiments, the present invention provides a water disinfecting system according to any one of the embodiments above, wherein said system further comprises a controller configured to receive input indicating at least one of: (a) one or more flow characteristics of the water (e.g., flow rate); (b) water temperature (e.g., using thermocouple); (c) intensity of light emission from said UV irradiation sources; (d) water turbidity; and (e) water UV transmittance, wherein based on said input, said controller controls/adjusts the energy input of said UV irradiation sources and/or the water flow rate to thereby optimize water disinfection.

The technology being the basis for the method and the apparatus 200 disclosed herein provides a low cost and highly-efficient solution for water disinfection. This technology is based on a combined, optionally simultaneous, use of a LP lamp, emitting a wavelength of about 254 nm that is absorbed by DNA molecules, and UV LEDs, emitting light at a wavelength of between 285 nm and 310 nm, and providing a synergistic effect to the irradiation effect of the LP lamp.

It should be understood, that although UVC-LEDs currently exist, such LEDs are expensive, and both their external quantum efficiency and wall-plug efficiency are very low (below 5% and 1-3%, respectively) in comparison to LP UV lamps (about 35% wall-plug efficiency; Beck et al., 2017), due to the low electrical conductivity of high aluminum composition AlGaN (aluminum gallium nitride). These limits make UVC-LEDs less practical as a replacement to LPUV lamps for the foreseen future (Moe, 2014). Indeed, the only UVC-LED based commercial water treatment device currently available is limited to a flow rate of 12 LPM, which is not realistic for any commercial scale water treatment that can be done using a combination of standard LDHF with matching LED lamps.

The term “about”, when used in this specification with respect to a wavelength, means that the wavelength value recited may vary by up to plus or minus 0.5 nm, e.g., a wavelength of about 254 nm may practically vary from 253.5 nm to 254.5 nm.

The invention will now be illustrated by the following non-limiting Examples.

### Examples

### Experimental Setup

### LP and MP Exposures

UV exposures were carried out using a medium pressure (MP) bench scale UV collimated beam apparatus. The UV irradiation was directed through a circular opening (collimated tube) to provide incident irradiation normal to the surface of the water test suspension. Emission spectrum of the LP and MP UV lamps are shown in FIG. 1. LP lamps emit UV irradiation with a maximum peak at −254 nm (253.7 nm), while MP lamps emit light at multiple peaks from 200 nm and above.

### Identifying Ranges of Wavelengths Facilitating Microorganism's Inactivation

Here we examine which specific wavelengths of UVA/B-LED range are the most effective for integration with LP or LPHO mercury lamp (or LED emitting light at a wavelength of about 254 nm) for inactivation and recovery control of various microorganisms.

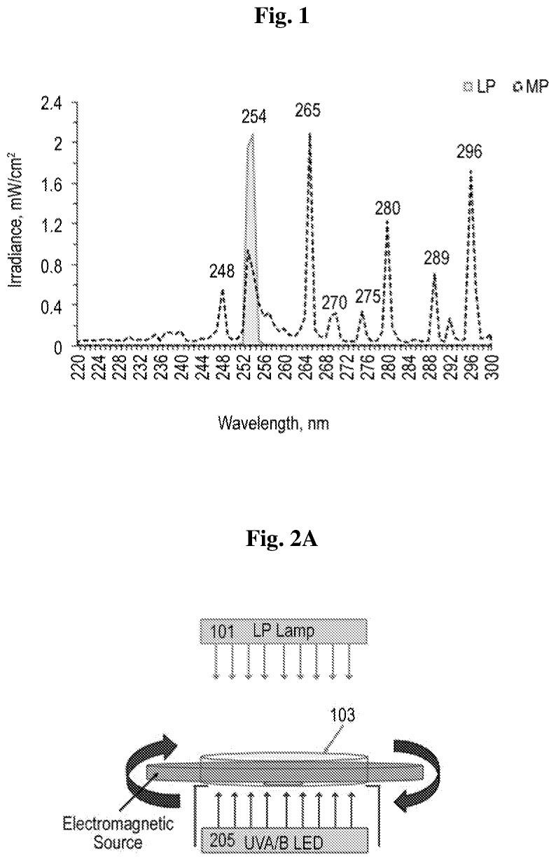

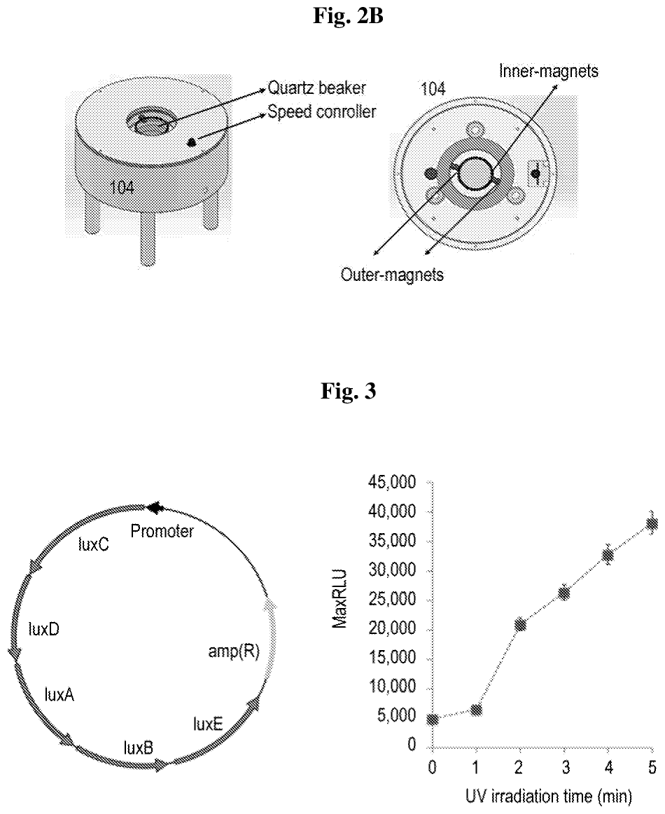

For this purpose, a unique LP-LED collimated beam apparatus was designed, that includes two UV sources emitting simultaneously: a mercury LP lamp 101 and a LED array 205, where the LP lamp irradiation is directed through a collimator and irradiates incident to the vessel 103 with a liquid containing microorganisms (a petri dish), and a UVA/B-LED system simultaneously irradiates from the other side of the petri dish 103 (FIG. 2A). To ensure mixing and uniformity, the liquid containing the microorganisms was continuously mixed using a unique stirring device 104 schematically shown in FIG. 2B, which allows mixing using a magnet that moves at the periphery thus not obscuring the irradiation from either side.

### Inactivation Experiments

To examine spiked E. coli and MS2 coliphage inactivation, aliquots of bacteria or bacteriophage were suspended in buffer at initial concentrations of about 10⁶ colony forming units (CFUs)/mL or plaque forming units (PFUs)/mL respectively. The microorganisms were exposed to a range of UV fluence by placing the suspension in a quartz dish and irradiating the samples while stirring. Irradiation was carried out using one or more of the following UV-sources: LP lamp and UV-LEDs. Each sample was serially diluted and enumerated to determine the bacterial (as CFUs) or bacteriophage number (as PFUs). Enumeration of spiked bacterial colonies was accomplished using the drop plate method (on LB plates) and enumeration of spiked bacteriophage plaques was accomplished using the double agar layer method (on tryptic soy agar (TSA) plates). Dose-response curves were developed by irradiating for different times, with microorganism concentration before UV exposure taken as the initial concentration, N₀, and arithmetic mean concentration per fluence as N_{D}. The log₁₀ transformation for N₀/N_{D} was plotted as a function of the average UV fluence.

The inactivation of secondary wastewater effluent containing indigenous bacteria was examined as well. To this end the water was placed in a quartz dish as above and irradiated with an LP, LED, or both, for the designated times. Water samples were taken, diluted (where needed) in sodium chloride saline solution and filtered on 0.2 μm filter. The filters were placed on m-ENDO agar LED plates and allowed to grow for 24 h at 35° C. For coliforms bacteria enumeration, red colonies with green metal sheen were counted.

For SodA promoter activation experiments, E. coli MG1655 carrying a plasmid with lux genes fused to the SodA promotor was used (FIG. 3). The bacteria were grown in Luria Bertani (LB) broth supplemented with the proper antibiotic, washed twice, and resuspended in phosphate buffered solution (PBS); and were then irradiated as above, and transferred to 96-well plate. Concentrated LB was added, and the bio-luminescence was quantified over 10 hours in plate reader. Increase in luminesce values indicated the sodA promoter activation.

### Results and Discussion

The efficiency of a hybrid system combining LP lamp with UVA/UVB wavelength from LEDs, as a replacement for MP lamps, was tested, wherein the LP lamp is used as a source for DNA-damaging 254 nm irradiation, and the UVA/B LED is used as a source for supplementary wavelength disinfecting the pathogens via different mechanisms such as production of radicals.

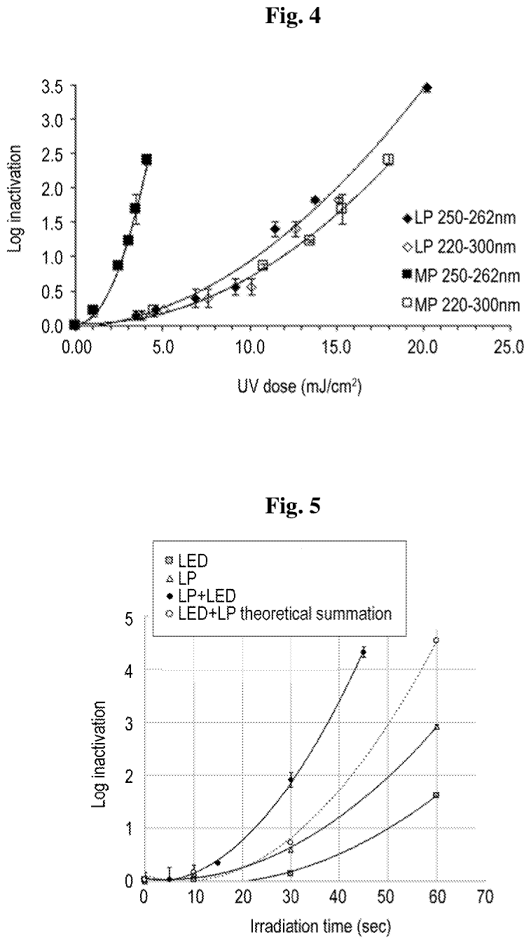

FIG. 4 shows the E. coli germicidal inactivation vs. irradiation dose for LP and MP lamp. When the irradiation was integrated between wavelengths of 250-262 nm (the range in which both lamps emit) the MP outperformed the LP, but when the 220-300 nm was integrated no benefit was noted for the MP over the LP. As the MP and LP have similar integrated irradiation at a wavelength range of 250-262 nm, the improved inactivation by the MP lamp demonstrates the importance of irradiation in wavelengths higher than the 262 nm range.

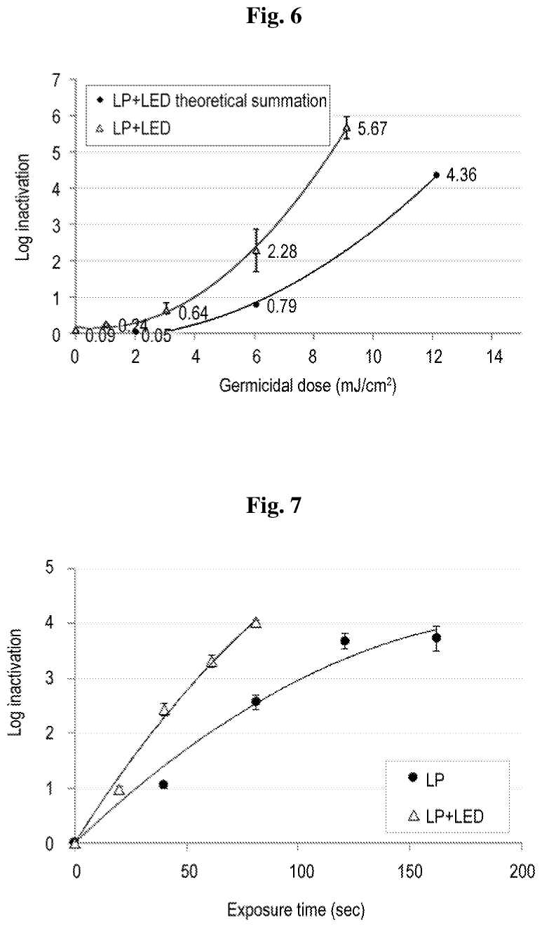

FIG. 5 and FIG. 6 show the inactivation curve of E. coli strain MG1655, by LP lamp or a UV LED emitting light in the 285-305 nm range, and the combination thereof, for various irradiation times and germicidal doses, respectively. The results demonstrate that the LP+LED combination results in enhanced and synergistic E. coli strain MG inactivation compared to either one of the two irradiation sources. Moreover, the germicidal efficiency of the combination was increased exponentially with increase in the irradiation time, in good agreement with involvement of more than one inactivation mechanism. The results shown illustrate that the combined LP+LED irradiation yields a maximum of 2.6 log over the theoretical additive effect of the two, and 3.5 log over LP only. Similar results were demonstrated for E. coli strain RFM443 (data not shown). The difference in inactivation vs. germicidal dose clearly demonstrates that mechanisms other than direct DNA damage are involved.

FIG. 7 shows log inactivation of natural indigenous coliforms in wastewater secondary effluent by LP or LP+LED emitting light in the 285-305 nm range. The data shown illustrate that the LP+LED combination results in enhanced inactivation compared to the LP irradiation alone also for indigenous microorganisms. Inactivation of Salmonella and/or Shigella in the same wastewater effluent provided similar results (data not shown).

In order to understand what happens inside the bacteria under UV irradiation, bacteria with reporter genes (lux) fused to different promoters were used. Among others, we used the sodA promoter, that promotes the expression of the superoxide dismutase enzyme that catalyzes destruction of O₂⁻ radicals and protects cells against harmful effects of superoxide radicals (Lee and Gu, 2003). As shown in FIG. 3, sodA gene was activated in E. coli bacteria exposed to MP lamp, indicating that exposure to MP lamp results in the formation of superoxide radical (O₂⁻ radicals) inside the bacteria, which could explain the more significant effect of MP lamps. Similarly to MP, irradiation with UV LED emitting light in the 285-305 nm range results in increase in the sodA promoter activation, suggesting that said irradiation generates superoxide radicals inside the cells (data not shown).

The results shown herein demonstrate that while photons from LP UV lamp target mainly DNA, irradiation of bacteria by photons at wavelengths longer than 254 nm, such as emitted from a MP UV lamp (in the UVB range), results also in the production of superoxide radicals inside the bacterial cell, most likely due to photoactivation of aromatic amino acids and mainly tryptophan. Such radicals can target cellular components other than DNA, e.g., cell membrane, small molecules, and proteins that are essential for almost all cell activities, including repair of DNA dimers. The combination of DNA targeting photons (such as from LP UV lamp) with superoxide generating photons (higher wavelength, such as from MP UV or UVA/B LED lamps) thus results in much more effective inactivation of pathogens and lower recovery thereof. Such a combined system has both the benefits of LP(OH) systems, i.e. low power consumption and low heat production, and of MP systems, i.e. better inactivation, and reduced recovery.

### REFERENCES

Beck, S. E.; Rodriguez, R. A.; Linden, K. G.; Hargy, T. M.; Larason, T. C; Wright, H. B. Wavelength dependent UV inactivation and DNA damage of adenovirus as measured by cell culture infectivity and long range quantitative PCR. Environmental science &technology, 2014, 48(1), 591-598
Beck, S. E.; Ryu, H.; Boczek, L. A.; Cashdollar, J. L.; Jeanis, K. M.; Rosenblum, J. S.; Lawal, O. R.; Linden, K. G. Evaluating UV-C LED disinfection performance and investigating potential dual-wavelength synergy. Water Res. 2017, 109, 207-216.
Beck, S. E.; Wright, H. B.; Hargy, T. M.; Larason, T. C.; Linden, K. G. Action spectra for validation of pathogen disinfection in medium-pressure ultraviolet (UV) systems. Water Research, 2015, 70, 27-37
Chen, R. Z.; Craik, S. A.; Bolton, J. R. Comparison of the action spectra and relative DNA absorbance spectra of microorganisms: Information important for the determination of germicidal fluence (UV dose) in an ultraviolet disinfection of water. Water Research, 2009, 43(20), 5087-5096
Chen, J.; Loeb, S.; Kim, J. H. Critical review: LED revolution: fundamentals and prospects for UV disinfection applications. Environ. Sci.: Water Res. Technol. 2017, DOI: 10.1039/c6ew00241b.
Detsch, R. M.; Bryant, F. D.; Coohill, T. P. The wavelength dependence of Herpes simplex virus inactivation by ultraviolet radiation. Photochemistry and photobiology, 1980, 32(2), 173-176
Gates F. L. A study of the bactericidal action of ultraviolet light. J Contam Hydrol. 1928, 13, 231-260
Gates, F. L. A study of the bactericidal action of ultra violet light III. The absorption of ultra violet light by bacteria. The Journal of general physiology, 1930, 14(1), 31-42
Harm, W. Biological effects of ultraviolet radiation. Press syndicate of the University of Cambridge, Cambridge, 1980
Jagger, J. Introduction to research in ultraviolet photobiology. Prentice-Hall, Eaglewood Cliffs, N. J., 1967
Lakretz, A.; Ron, E. Z.; Mamane, H. Biofouling control in water by various UVC wavelengths and doses, Biofouling, 2010, 26, 257-267
Lee, H. G.; Gu, M. B. Construction of a sodA:luxCDABE fusion Escherichia coli: comparison with a katG fusion strain through their responses to oxidative stresses, Appl. Microbiol. Biotechnol., 2003. 60, 577-580
Linden, K.; Shin, G. A.; Sobsey, M. D. Relative efficacy of UV wavelengths for the inactivation of Cryptosporidium parvum. Water Science and Technology, 2001, 43(12), 171-174
Mamane-Gravetz, H.; Linden, K. G.; Cabaj, A.; Sommer, R. Spectral sensitivity of Bacillus subtilis spores and MS2 coliphage for validation testing of ultraviolet reactors for water disinfection. Environmental science &technology, 2005, 39(20), 7845-7852
Meulemans, C. C. E. The basic principles of UV-disinfection of water, 1987
   Moe, C. UV-C light emitting diodes, Radtech Report, 2014, 1, 45-49
   Oguma, K.; Katayama, H.; Ohgaki, S. Photoreactivation of Escherichia coli after low- or medium-pressure UV disinfection determined by an endonuclease sensitive site assay. Appl. Environ. Microbiol. 2002, 68(12), 6029-6035
Sinha, R. P.; Hader, D. P. UV-induced DNA damage and repair: a review. Photochem. Photobiol. Sci. 2002, 1, 225-236
USEPA, 2006. Ultraviolet disinfection guidance manual for the final long term 2 enhanced surface water treatment rule, Environmental Protection. EPA 815-R-06-007
Wang, T.; MacGregor, S. J.; Anderson, J. G.; Woolsey, G. A. Pulsed ultra-violet inactivation spectrum of Escherichia coli. Water research, 2005, 39(13), 2921-2925
Water Environment Federation (WEF), 2015. Ultraviolet disinfection process concepts and equipment systems, in ultraviolet disinfection for wastewater. p. 17-29
Zimmer, J. L.; Slawson, R. M. Potential repair of Escherichia coli DNA following exposure to UV radiation from both medium- and low-pressure UV sources used in drinking water treatment. Appl. Environ. Microbiol. 2002, 68(7), 3293-3299

1. A method for water disinfection comprising exposing water to a combination of at least two ultraviolet (UV) irradiation sources, for a sufficient period of time, wherein at least one of said UV irradiation sources emits light at a wavelength of between 250 nm and 280 nm, and at least one of said UV irradiation sources emits light at a wavelength of between 285 nm and 310 nm.

2. The method of claim 1, wherein:
(i) said at least one UV irradiation source emitting light at a wavelength of between 250 nm and 280 nm comprises a sole UV irradiation source or a plurality of UV irradiation sources emitting light at either the same or different wavelengths between 250 nm and 280 nm; or
(ii) said at least one UV irradiation source emitting light at a wavelength of between 285 nm and 310 nm comprises a sole UV LED irradiation source or a plurality of UV LED irradiation sources emitting light at either the same or different wavelengths between 285 nm and 310 nm.

3. The method of claim 2, wherein said at least one UV irradiation source comprises a low-pressure (LP) UV irradiation source, or at least one UV light-emitting diode (LED) irradiation source each independently emitting light at a wavelength of between 260 nm and 275 nm, between 260 nm and 270 nm, or between 260 nm and 265 nm.

4. The method of claim 3, wherein said LP UV irradiation source emits light at a wavelength of about 254 nm.

5-6. (canceled)

7. The method of claim 2, wherein each one of said UV LED irradiation sources independently emits light at a wavelength of between 285 nm and 290 nm, between 290 nm and 295 nm, between 295 nm and 300 nm, between 300 nm and 305 nm, or between 305 nm and 310 nm.

8. (canceled)

9. The method of claim 1, wherein:
(i) said at least one UV irradiation source emitting light at a wavelength of between 250 nm and 280 nm comprises: (a) a LP UV irradiation source emitting light at a wavelength of about 254 nm; or (b) a UV LED irradiation source emitting light at a wavelength of between 260 nm and 275 nm, between 260 nm and 270 nm, or between 260 nm and 265 nm; and
(ii) said at least one UV irradiation source emitting light at a wavelength of between 285 nm and 310 nm comprises a UV LED irradiation source emitting light at a wavelength of between 285 nm and 290 nm, between 290 nm and 295 nm, between 295 nm and 300 nm, between 300 nm and 305 nm, or between 305 nm and 310 nm.

10. (canceled)

11. The method of claim 9, wherein the exposure of said water to said at least two UV irradiation sources is carried out simultaneously.

12. A water disinfecting apparatus comprising:
(i) a chamber having an inlet adapted for connection to a pressurized water source, and an outlet;
(ii) at least one ultraviolet (UV) irradiation source emitting light at a wavelength of between 250 nm and 280 nm; and
(iii) at least one UV irradiation source emitting light at a wavelength of between 285 nm and 310 nm,
wherein said UV irradiation sources are designed/configured to emit UV light into said chamber when water passes therethrough.

13. The apparatus of claim 12, wherein:
(i) said at least one UV irradiation source emitting light at a wavelength of between 250 nm and 280 nm comprises a sole UV irradiation source or a plurality of UV irradiation sources emitting light at either the same or different wavelengths between 250 nm and 280 nm; or
(ii) said at least one UV irradiation source emitting light at a wavelength of between 285 nm and 310 nm comprises a sole UV LED irradiation source or a plurality of UV LED irradiation sources emitting light at either the same or different wavelengths between 285 nm and 310 nm.

14. The apparatus of claim 13, wherein said at least one UV irradiation source comprises a low-pressure (LP) UV irradiation source, or at least one UV light-emitting diode (LED) irradiation source each independently emitting light at a wavelength of between 260 nm and 275 nm, between 260 nm and 270 nm, or between 260 nm and 265 nm.

15. The apparatus of claim 14, wherein said LP UV irradiation source emits light at a wavelength of about 254 nm.

16-17. (canceled)

18. The apparatus of claim 13, wherein each one of said UV LED irradiation sources independently emits light at a wavelength of between 285 nm and 290 nm, between 290 nm and 295 nm, between 295 nm and 300 nm, between 300 nm and 305 nm, or between 305 nm and 310 nm.

19. The apparatus of claim 12, wherein:
(i) said at least one UV irradiation source emitting light at a wavelength of between 250 nm and 280 nm comprises (a) LP UV irradiation source emitting light at a wavelength of about 254 nm; or (b) a UV LED irradiation source emitting light at a wavelength of between 260 nm and 275 nm, between 260 nm and 270 nm, or between 260 nm and 265 nm; and
(ii) said at least one UV irradiation source emitting light at a wavelength of between 285 nm and 310 nm comprises a UV LED irradiation source emitting light at a wavelength of between 285 nm and 290 nm, between 290 nm and 295 nm, between 295 nm and 300 nm, between 300 nm and 305 nm, or between 305 nm and 310 nm.

20. The apparatus of claim 19, wherein:
(i) said at least one UV irradiation source emitting light at a wavelength of between 250 nm and 280 nm comprises (a) LP UV irradiation source emitting light at a wavelength of about 254 nm; or (b) a UV LED irradiation source emitting light at a wavelength of between 260 nm and 270 nm; and
(ii) said at least one UV irradiation sources emitting light at a wavelength of between 285 nm and 310 nm comprises a UV LED irradiation source emitting light at a wavelength of between 290 nm and 300 nm.

21. The apparatus of claim 12, wherein said chamber is configured as a tube.

22. The apparatus of claim 21, wherein:
(i) said at least one UV irradiation source emitting light at a wavelength of between 250 nm and 280 nm is located across the center of said tube; and
(ii) said at least one UV irradiation source emitting light at a wavelength of between 285 nm and 310 nm is a UV light-emitting diode (LED) located at the perimeter of said tube.

23. The apparatus of claim 22, wherein:
(i) said at least one UV LED irradiation source emitting light at a wavelength of between 285 nm and 310 nm is distributed around and across the perimeter of said tube, or located inside said tube and is waterproof; or
(ii) said at least one UV irradiation source emitting light at a wavelength of between 250 nm and 280 nm is located in a transparent sleeve located at the center along the axis of said tube.

24. The apparatus of claim 22, wherein said tube is either made of, or comprises at least one region made of, a material transparent to said UV LED irradiation of a wavelength of between 285 nm and 310 nm but not transparent to said LP UV irradiation of a wavelength of between 250 nm and 280 nm; and said at least one UV LED irradiation source emitting light at a wavelength of between 285 nm and 310 nm is located externally to said tube or said at least one region, respectively, such that it does not come in contact with the water when passes through said tube.

25-26. (canceled)

27. The apparatus of claim 23, wherein said transparent sleeve is made of quartz, soda lime glass, or a UVC-transparent polymer such as a polyacrylate.

28. The apparatus of claim 22, wherein:
(i) said at least one UV LED irradiation source emitting light at a wavelength of between 285 nm and 310 nm is distributed around and across the perimeter of said tube, and is located externally such that it does not come in contact with the water when passes through said tube; and
(ii) said at least one UV irradiation source emitting light at a wavelength of between 250 nm and 280 nm is located in a transparent sleeve located at the center along the axis of said tube, wherein said transparent sleeve is made of quartz or a UVC-transparent polymer.

29. The apparatus of claim 21, wherein said tube further comprises:
(i) reflective surface(s) for increasing the effect of UV irradiation on the water when passes through said tube; and/or
(ii) baffles to enhance whirling of water when passes through said tube, thus increasing the time during which said water is being exposed to said UV irradiation.

30. The apparatus of claim 29, wherein said reflective surface(s) are made of polytetrafluoroethylene (Teflon), aluminum, stainless steel, or a refractive polymer, optionally coated for better reflectance.

31. (canceled)

32. The apparatus of claim 12, further comprising a controller configured to receive input indicating at least one of:
(a) one or more flow characteristics of the water;
(b) water temperature;
(c) intensity of light emission from said UV irradiation sources;
(d) water turbidity; and
(e) water UV transmittance,
wherein based on said input, said controller controls/adjusts the energy input of said UV irradiation sources and/or the water flow rate to thereby optimize water disinfection.
